(19) United States
(12) Patent Application Publication
(10) Pub. No.: US 2009/0223531 A1
(43) Pub. Date: Sep. 10, 2009

(54) Applicator for a Hair Treatment Composition

(76) Inventors: Mark Thomas Lund, Sunningdale (GB); Paul James Smith, Twickenham (GB)

Correspondence Address: THE PROCTER & GAMBLE COMPANY;Global Legal Department - IP, Sycamore Building - 4th Floor, 299 East Sixth Street, CINCINNATI, OH, 45202, US

(21) Appl. No.: 12/135,289

(22) Filed: Jun. 9, 2008

(30) Foreign Application Priority Data
Jun. 15, 2007 (EP) 07110395.6

(51) Int. Cl. A61K 8/00; A45D 19/00

(52) U.S. Cl. 132/208; 132/270

(57) ABSTRACT

The present invention relates to an applicator (1) which allows for precise, non-messy and even application of a hair treatment composition to a hair strand. The applicator (1) comprises a plate (10) movably joined by a connection (30) to a well (20), a hair orientation means on the internal surface (101) of the plate (10) which extends towards the well (20) and a fluid metering means (3) comprising a first metering layer (50) on the rim (222) of the well (20) and a second metering layer (60) on the internal surface of said plate (10).

### FIELD OF THE INVENTION

The present invention relates to an applicator which allows for precise, non-messy and even application of a cosmetic composition to fibres, preferably keratinous fibres. The applicator, which comprises a hair orientation means and a fluid metering means, is especially intended for a hair treatment composition to provide hair strand effects.

### BACKGROUND OF THE INVENTION

Application of hair treatment compositions to distinct hair strands allows the user to achieve a different look than a full head application. Hair treatment compositions for providing a hair strand effects include highlighting compositions, dyeing compositions, perming compositions, styling compositions and mixtures thereof.

Hair strand effects such as those provided by highlighting compositions and dyeing compositions must be precisely applied where desired. For example, if a too abundant amount of highlighting composition is applied to the root, it may transfer to the neighbouring unselected hair strands. This may alter the overall end result and may totally disrupt the pattern that the user has tried to create. If an excessive amount of product is applied to the root, the colour effect will not be consistent along the length of the hair, leading to an undesired visual effect. If, instead, insufficient composition is applied to the hair strands, the evenness of the hair strand effect may not be achieved producing an end result which is visually unacceptable. Hence it is important that a consistent amount of product is applied uniformly along the hair strands being treated.

One known method for providing hair strand effects such as highlighting is the cap and hook system. A cap, provided with holes, is positioned over the head and hair strands are pulled out with a hook. Far from being accurate, the cap and hook system suffers from several drawbacks including random selection of the hair strands via the holes on the cap and the likelihood of applying the highlighting composition to only a portion of the selected hair strands and not to the root portion.

Several applicators have been designed for application of a hair treatment composition to independent bundles of hair strands as alternatives to the cap and hook system. These applicators belong to two general fields. One field comprises applicators based on combs and/or brushes. The other group comprises applicators having two articulated portions which are movable one relative to the other. Many attempts have been disclosed in this later field. U.S. Pat. No. 3,030,968 refers to an applicator for liquid treating material to be loaded by immersion. This applicator comprises a trough and a hair guide member mounted on the ends of the legs of a U-shaped resilient spring. The spring allows for manual compression and permits the hair guide member to fit into the trough. U.S. Pat. No. 6,062,231 discloses a device for applying a hair product to hair strands. This device comprises two articulated portions; the application means to be loaded by immersion and the retaining member to keep the hair strands on the applicator means while the device is in use. Another attempt is shown in US2003/0024544 wherein a device is disclosed provided with a cavity for the hair product and a retention member which is elastically deformable. The retention member may comprise porous or fibrous material and the cavity is provided with at least one notch to keep the hair strands in position during the application of the hair treatment composition. However the use of such members has a number of drawbacks, in particular the deposition of the composition is not uniform across all the hair strands and or may excessively coat the hair or not coat some hair strands at all. Moreover, the composition may also be displaced from the applicator causing mess.

It is generally recognized that the self-application of a composition to achieve hair strand effects are difficult per se, in particular those for highlighting and dyeing. To achieve the expected end results, an applicator capable of facilitating the self-application of a hair treatment composition needs to be conceived to address several technical challenges but in particular the applicator should evenly apply the composition to independent bundles of hair strands. Evenness is very important when the composition is a highlighting or dyeing composition. The permanent effect provided by these compositions is not immediately visible after the application and if the result is not appealing, it is not easily reversed. An applicator should hence ensure homogeneous coating along the length and width of the bundle of hair strands and likewise on the front and rear surfaces.

In addition, such applicators should apply an amount of hair treatment composition, which is sufficient to coat all of the hair strands and thereby provide a hair strand effect without transferring to neighbouring strands or the scalp and skin. Furthermore, the applicator should not apply but then subsequently scrape off the hair treatment composition while the user moves the applicator along the bundle of hair strands. The application with such an applicator should also occur in a tidy and clean fashion without the hair treatment composition leaking out of the hair treatment applicator. The applicator should also allow the movement of hair through the applicator without hindering its path resulting in snaring, entanglement and potentially discomfort to the user.

Finally, such an applicator for hair treatment compositions should be easy to use; it should be doubtless cheap and easy to produce and it should not require any special experience and training in matters such as how much and where to load the hair treatment composition. Ideally, the consumer should be able to load and use the applicator by simply following a few instructions provided by the manufacturer.

Thus, what still remains to be solved in the art is a hair treatment applicator capable of overcoming the technical problem defined above.

It has now been found that an applicator (as defined herein after) can significantly improve the application of a hair treatment composition to provide hair strand effects.

### SUMMARY OF THE INVENTION

According to the invention, an applicator (1) for applying a hair treatment composition to the hair is provided, said applicator (1) comprising a plate (10) and a well (20); wherein said plate (10) and well (20) are movably joined by a connection (30) so that the applicator (1) may alternate between a closed and an open state, and wherein said plate (10) has an external surface (102) and an internal surface (101), and said internal surface (101) comprises at least one hair orientation means which extends beyond said internal surface (101) towards said well (20), and

wherein said well (20) comprises a rim (222) and wherein said applicator (1) further comprises a fluid metering means (3), wherein said fluid metering means (3) comprises a first metering layer (50) positioned on said rim (222) of said well (20), and a second fluid metering layer (60) positioned on said internal surface (101) of said plate (10), such that when said applicator (1) is in said closed state said first metering layer (50) and said second metering layer (60) are substantially juxtaposed to provide said fluid metering means.

Furthermore, a method to apply a hair treatment composition to a hair strand whereby said hair strand is contacted with said applicator (1) according to the invention and a kit-of-parts comprising an applicator (1) according to the invention is also described.

### BRIEF DESCRIPTION OF THE DRAWINGS

FIG. 1A is a perspective view of an embodiment of said applicator (1) according to the invention. Said applicator comprises a plate (10), a well (20) and a connection (30). In this embodiment, a first metering layer (50), is laid upon said rim (222) of said wall (202) of said well (20) and a second metering layer (60) is laid upon the internal surface (101) of said plate (10) along said perimeter (103) of said plate (10) and a hair orientation means which is a member (40) that projects from said internal surface (101) of said plate (10). The applicator (1) is shown herein in an open state, whereby said internal surface (101) of said plate (10) is in a distant relationship to said opening (203) of said well (20). On said rim (222) two stop mechanisms, here represented by two substantially identical hemispheres (402; 403), are shown. On the bottom (201) of said well (20) adjacent to said wall (202) at the side of the connection (30) said applicator (1) comprises a sealing means (401).

FIG. 1B shows a perspective view of an embodiment of the applicator (1) according to the invention comprising a plate (10) and a well (20). In this embodiment said at least one metering layer comprises a first metering layer (50), which is laid upon said rim of said wall (202) of said well (20) and a second metering layer (60) laid upon said internal surface (101) of said plate (10) along said perimeter (103) of said plate (10). A hair orientation means comprising a member (40) projects from said internal surface (101) of said plate (10).

FIG. 2A is a perspective view of an embodiment of the applicator (1) according to the invention. The applicator (1) is shown in this embodiment in a closed state. Part of said wall (202) of said well (20) has been removed to show that from said internal surface (101) of said plate (10), which in this embodiment is substantially flat, two substantially identical first fin (70) and second fin (71) project in a way that said proximal edges (90; 91) are substantially parallel to said axis Y and said distal edges (80; 81) verge one toward the other. A first metering layer (50) is laid upon said rim (222) of said wall (202) and a second metering layer (60) is laid upon said substantially flat internal surface (101), along said perimeter (103) of said plate (10).

FIG. 2B is a cross-section of said plate (10) shown in FIG. 2A. The cross-section is taken perpendicular to said axis Y, along line AB in FIG. 2A. Said two substantially identical first and second fins (70; 71) extend for substantially identical average first and second lengths (L1; L2) and form with said substantially flat internal surface (101) substantially identical angles α and β.

FIG. 2C is the same cross-section as shown in FIG. 2B but evidencing said average distance (D1) between said perimeter (103) of said plate (10) and said rim (222) of said wall (202). Said first metering layer (50) and said second metering layer (60) are also shown.

FIG. 3 is a perspective view of an embodiment of said applicator (1) according to the invention. Said plate (10) is movably joined to said well (20) by a connection (30). A member (40), which has a substantially pyramidal frustum form, projects from said internal surface (101) of said plate (10). On said rim (222) a first metering layer (50) part of which has been removed to show the rim (222) is laid upon said rim (222). A second metering layer (60) is laid upon the internal surface (101) of said plate (10).

FIG. 4 is a perspective view of an embodiment of an applicator (1) according to the invention. Said applicator (1) comprises a plate (10) connected by a connection (30) to a well (20). Said connection (30) comprises two female parts (32; 34) fixed by a pin (38). Two strips (11; 12) are placed on said rim (222) of said wall (202) of said well (20). A first metering layer is laid upon said rim (222) in two pieces (50; 50′). A second metering layer is laid upon said internal surface (101) along said perimeter (103) in two pieces (60; 60′). Two substantially identical slots (49; 59) are cut through said plate (10) to accommodate two substantially identical (70; 71) fins.

FIGS. 5A, 5B and 5C are cross-sectional views of said plate (10) of said applicator (1) according to the invention. The cross-sectional views are taken at the centre of said plate (10) along axis Y. One or more dipping means project from said internal surface (101) of said plate (10). Said one or more dipping means are: in FIG. 5A a member (40) having a substantially pyramidal form; in FIG. 5B a plurality of tines (41); in FIG. 5C a member (40) having a substantially parallelepiped form.

FIGS. 5D, 5E, 5F and 5G are cross-sectional views of a plate (10) of an embodiment of said applicator (1) according to the invention. The cross-sectional views are taken transversally to said axis Y (not shown herein) and substantially at the centre of said plate (10). Each view shows a plate (10) comprising a first (70) and a second (71) fin, wherein said fins (70; 71) have different shapes.

FIGS. 5H, 5L and 5M are cross-sectional views of a plate (10) of an embodiment of said applicator (1) according to the invention. The cross-sectional views are taken transversally to said axis Y (not shown herein) and substantially at the centre of said plate (10). Each view shows a plate (10) comprising a first (70) and a second (71) fin, wherein each of said fins (70; 71) has a substantially rectangular shape. Said internal surface (101) of said plate (10) is: in FIG. 5H waved, in FIG. 5L concave and in FIG. 5M is convex.

FIGS. 5N and 5P are perspective views of the internal surface (101) of a plate (10) of an embodiment of said applicator (1) according to the invention. A first (70) and a second (71) fin project from said internal surface (101) of said plate (10), in FIG. 5N said fins (70; 71) and their proximal edges (90; 91) are curved, whilst in FIG. 5P said fins (70; 71) have protrusions (75).

### DETAILED DESCRIPTION OF THE INVENTION

For the purpose of this invention, the term hair refers to both living hair i.e. on a living body and to non-living hair i.e. in a wig, hairpiece or other aggregation of non-living keratinous fibre. Mammalian, preferably human hair is intended.

For the purpose of this invention, the term “laid upon” is generally used to indicate the location of the feature to which it refers and not the act of locating it.

The present invention is characterized by the synergistic relationship that the features as described herein have when combined together in the specific relationship selected within the present invention to solve the above technical problem.

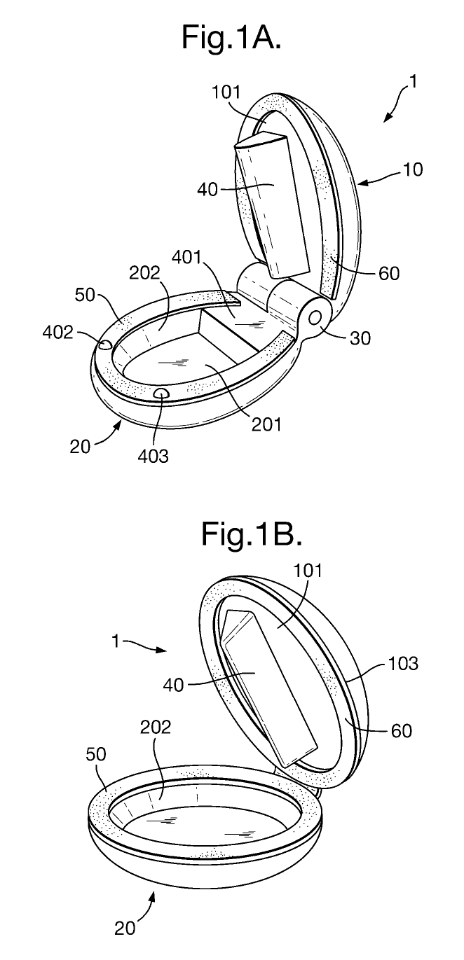

To achieve the technical effect described herein, the applicator (1) for applying a hair treatment composition to the hair according to the present invention comprises a plate (10) and a well (20) as shown in FIG. 1A. A connection (30) movably joins said plate (10) to said well (20). Said plate (10) comprises a perimeter (103) and an internal (101) and an external surface (102). Said well (20) is formed by a bottom (201) and a wall (202), said wall (202) emerging from said bottom (201) and extending upwardly. Said wall (202) comprises a rim (222), said wall (202) and said rim (222) define an opening (203) and an internal volume (204) of said well (20) as shown in FIG. 1. Said plate (10) and said well (20) are movably joined by said connection (30), so that said applicator (1) can alternate from an open state to a closed state. In this latter state, said internal surface (101) of said plate (10) is in a juxtaposed relationship to said opening (203) of said well (20).

Said internal surface (101) of said plate (10) further comprises at least one hair orientation means, which in FIG. 1A is a member (40) which extends beyond said internal surface (101) towards said well (20), preferably towards but not touching said bottom (201) of said well (20).

Said applicator (1) further comprises a fluid metering means. Preferably said fluid metering means is selected from the group consisting of non-wovens, foams, and combinations thereof. Preferably said metering means has a caliper of from about 0.40 mm to about 21.88 mm and a compressibility as defined herein of from about 59% to about 93%.

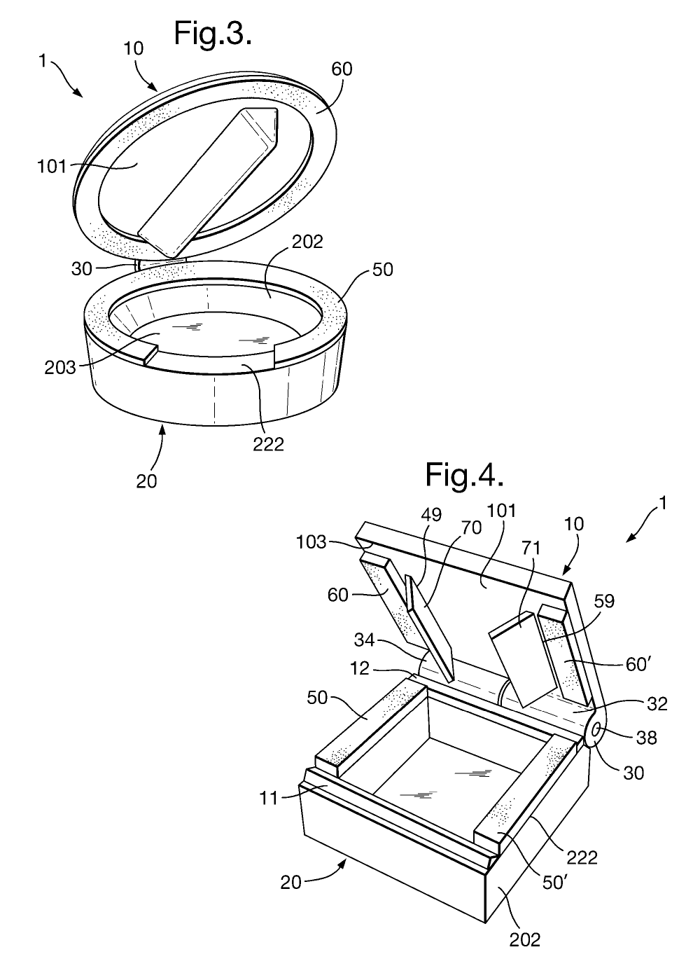

Said fluid metering means comprises a first metering layer (50) and a second metering layer (60). Said first metering layer (50) is laid upon said rim (222) of said well (202) of said well (20) as seen in FIGS. 1A, 1B and 3.

Said second metering layer (60) is laid upon said internal surface (101) of said plate (10), preferably along said perimeter (103) of said plate (10). Said first metering layer (50) and said second metering layer (60) are positioned such that when said applicator (1) is in said closed state, said first metering layer (50) and said second metering layer (60) are substantially juxtaposed to provide said fluid metering means.

The combination of said hair orientation member (2) and fluid metering means comprising a first metering layer (50) and second metering layer (60) as described below enable said applicator (1) to perform an application of a hair treatment composition to a hair strand, not only in a clean and non-messy fashion, but also evenly to provide an effective hair strand effect.

### 1. Applicator

The applicator (1) according to the present invention comprises a plate (10) movably joined to a well (20). Said plate (10) and said well (20) of said applicator (1) according to the invention are of ergonomic size and can thus fit easily on either hand. Said internal surface (101) preferably has a surface area of from about 2 cm² to about 150 cm², preferably from about 2 cm² to about 70 cm², more preferably from about 3 cm² to about 50 cm² and even more preferably from about 4 cm² to about 30 cm². The shape of said plate (10) may vary. Rectangular, square, circular, elliptical, oblong shape or combination thereof may be useful as they are easy to manufacture but other shapes, particularly those that are easily recognized by the consumers may also be used.

Said plate (10) of said applicator (1) comprises an axis Y. Axis Y extends straight from the centre of said plate (10) and transversally crosses said connection (30), preferably substantially perpendicular to said connection (30).

Said plate (10) comprises a perimeter (103), an internal surface (101) and an external surface (102). Said well (20) comprises a bottom (201), a wall (202) and said wall (202) comprises a rim (222). Said rim (222) defines an opening (203) and an internal volume (204) of said well (20). Said internal volume (204) is preferably for containing a hair treatment composition.

Preferably, said perimeter (103) of said plate (10) and said rim (222) of said wall (202) of said well (20) may be curvilinear or sharp. Said perimeter (103) of said plate (10) and said rim (222) of said well (202) of said wall (20) have each independently a length. Preferably, said perimeter (103) and said rim (222), have substantially identical lengths. Said rim (222) also comprises a width. Preferably, said width of said rim (222) is from about 1 mm to about 20 mm, more preferably from about 2 mm to about 15 mm, even more preferably from about 3 mm to about 8 mm.

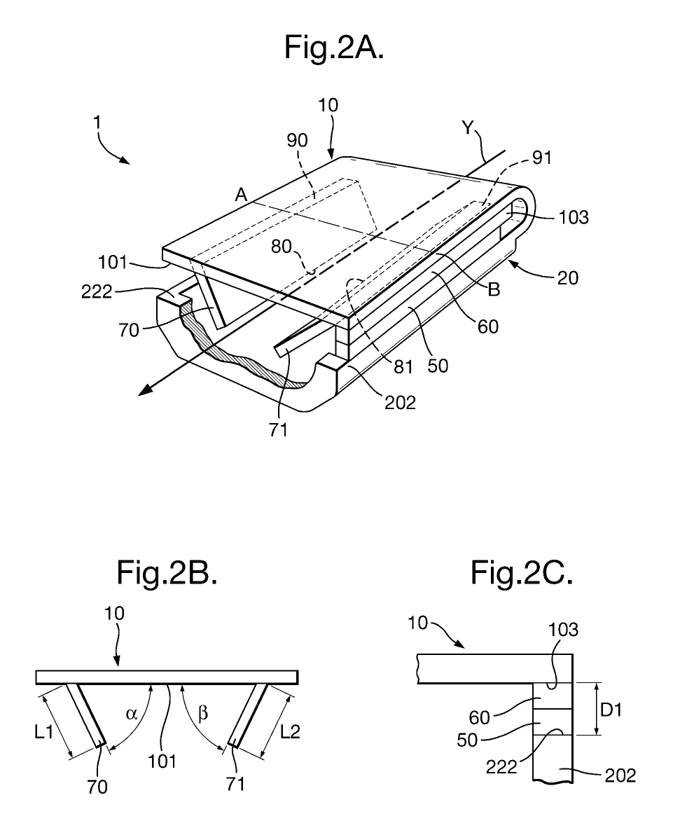

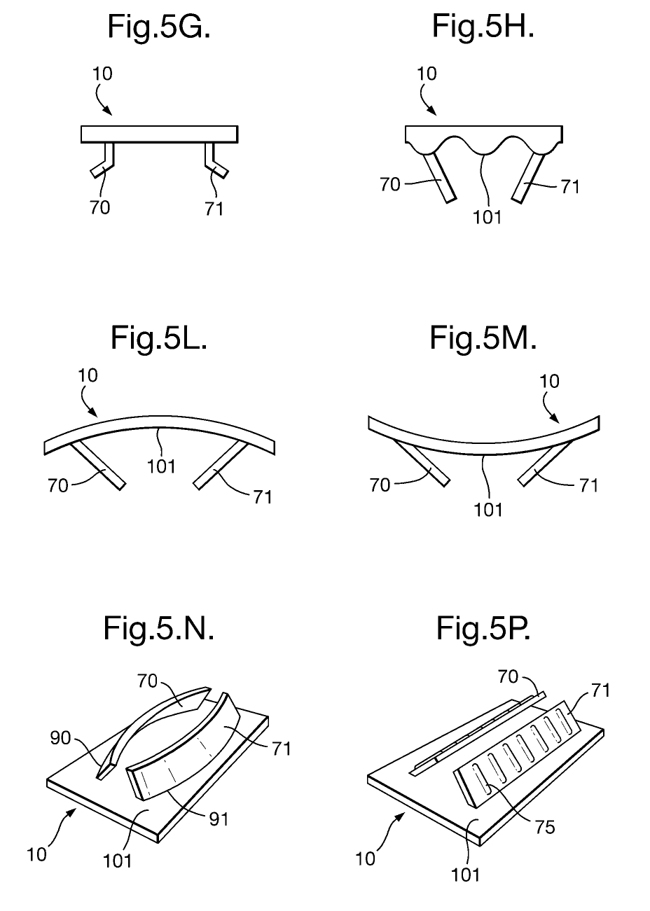

Said internal surface (101) of said plate (10) may be substantially flat as shown in FIG. 2A or may be concave as shown in FIG. 5L or may be convex as shown in FIG. 5M. In addition said internal surface (101) may have a waved pattern as shown in FIG. 5H. Said external surface (102), said wall (202) and said bottom (201) may also be substantially flat, concave, convex or waved. Preferably, said bottom (201) of said well (20) is substantially flat.

Said plate (10) and said well (20) may be manufactured from any known material or combination of materials capable of supporting a hair treatment composition. Suitable materials are polymer resins such as a polyolefins e.g. polypropylene, polyethylene or polyethylene terephthalate. Other materials which could be used include polyvinylchloride, polyamide, acetyl, acrylonitrile butadiene styrene, acrylic, acrylonitrile styrene acrylate, ethylene vinyl alcohol, polycarbonate, polystyrene, silicone or thermo plastic elastomer, thermo plastic vulcanate or copolymers where appropriate; flexible pliable substrates such as paper boards, metal based substrates and aluminium foils, filmic substrates or multiple laminations or combinations of multiple layers of said materials.

The method of manufacture of said plate (10) and said well (20) may include, but is not limited to, injection moulding, co-injection moulding, over moulding, in-mold assembly, compression moulding, blow moulding, thermo or vacuum forming of a blister type shell and lamination onto a carrier plastic or board material in the horizontal or vertical plane.

A connection (30) movably joins said plate (10) and said well (20) such that it is possible for said applicator (1) to alternate from an open state to a closed state. Said applicator (1) is shown in FIG. 2A in a closed state. When said device (10) is in a closed state, said internal surface (101) of said plate (10) is in a juxtaposed relationship to said opening (203) of said well (20). When said applicator (1) is in a closed state, said perimeter (103) of said plate (10) has an average distance (D1) from said rim (222) of said wall (20) of said well (20). Said average distance (D1) is preferably from about 0.5 mm to about 5.0 mm, more preferably from about 0.8 mm to about 4.0 mm, even more preferably from about 1.0 mm to about 3.0 mm. The average distance (D1) was determined using a Mitutoyo Digimatic calipers as described hereafter.

Said plate (10) and said well (20) are connected via any suitable means that fulfils the above described requirements for a connection (30), including the user's hand, for example through the thumb and index finger. In one embodiment, said plate (10) and said well (20) are mounted at the ends of the arms of a tweezers-like or tong-like connection (30). In another embodiment said plate (10) and said well (20) are connected via one or more hinges, preferably one hinge. Preferably, said connection (30) is contiguous and located adjacent to said perimeter (103) of said plate (10) and to said rim (222) of said well (20).

A connection (30) is necessary to improve the user's perception of control over the applicator (1) and to allow the user to guide the applicator (1), with the use of either hand, precisely and easily to each bundle of hair strands.

### 2. Hair Orientation Means

The applicator (1) of the present invention further comprises at least one hair orientation means. Said hair orientation means projects from said internal surface (101) of said plate (10). The presence of said hair orientation means ensures that the hair strand is bent within said well (20). Without wishing to be bound by theory it is believed that the hair orientation means enables said hair strand, preferably said bundle of hair strands, to contact said hair treatment composition within said internal volume (204) of said well (20) and not only at said opening (203) of said well (20). This improves the evenness of the application, in particular, the evenness from the root to the tip of the bundle of hair strands as described herein after.

In one embodiment of the present invention, said hair orientation means is a member (40), which projects from said internal surface (101) of said plate (10), wherein said member (40) has preferably a substantially pyramidal frustum form, as shown in FIG. 3. Said member (40) may have various forms including, but not limited to, a parallelepiped form, a cube form, a cylinder form, a conical or a pyramidal form as shown in FIG. 5A. Said member may have a substantially parallelepiped form as shown in FIG. 5C. Said member (40) may also be composed of a plurality of independent units grouped together; said independent units may comprise bristles, teeth or tines (41) as shown in FIG. 5B. Said member (40) has preferably a substantially pyramidal frustum form as shown in FIG. 3. In another embodiment not show herein, said member (40) comprises a cylinder which is rotary engaged via its circular bases to said internal surface (101) via two pins, emerging from said internal surface (101) of said plate (10).

Said member (40) projects from said internal surface (101) of said plate (10) with a maximum height (H) and extends along said internal surface (101) with a maximum width (W) and a maximum length (L). Said member (40), preferably projects orthogonally to said axis Y with said maximum height (H). Said member (40) may extend along said internal surface (101) of said plate (10) with its maximum length (L) either along said axis Y or substantially parallel to said axis Y or transversally to said axis Y.

Preferably, said maximum length (L) is at least twice said maximum width (W). The maximum length (L) is preferably from about 20.0 cm to about 0.2 cm, more preferably from about 15.0 cm to about 0.3 cm, even more preferably from about 10.0 cm to about 0.5 cm. The maximum width (W) is preferably from about 2.5 cm to about 0.01 cm, more preferably from about 1.0 cm to about 0.02 cm, even more preferably from about 0.5 cm to about 0.03 cm. The maximum height (H) is preferably from about 5.0 cm to about 0.1 cm, more preferably from about 2.5 cm to about 0.2 cm, even more preferably from about 1.5 cm to about 0.3 cm.

When said applicator (1) is in a closed state, said member (40) does not contact said bottom (201) of said well (20), so that a passage is left and said hair strand, preferably said bundle of hair strands, is not constrained.

In another embodiment according to the invention said hair orientation means comprises a first fin (70), and preferably a first (70) and a second fin (71), which independently project from said internal surface (101) of said plate (10) as shown in FIG. 2A. The term “fin” within the scope of the present invention defines a strip or sheet of material, preferably of substantially constant thickness as described below. The form of said first (70) and/or second (71) fin may vary; preferably said first (70) and/or second (71) fin have the form of a parallelepiped wherein two of the six faces extend for an area which is at least twice the area of the other four faces. Those two faces have preferably a substantially flat surface. The shape of said first (70) and second (71) fin may vary. Rectangular, square, circular, elliptical, oblong or combination thereof may be useful. A rectangular shape as shown in FIG. 2A is preferred. Other shapes and forms of said first and second fins (70; 71) may be used to bend said hair strand within said internal volume (204) of said well (20). Some examples are given in FIGS. 5D, 5E, 5F and 5G.

Said first fin (70) projects from said internal surface (101) of said plate (10) and extends for an average first length (L1) of from about 2 mm to about 30 mm. Said first fin (70) forms with said internal surface (101) of said plate (10) and angle α of from about 15° to about 75°. Preferably, when said internal surface (101) of said plate (10) is substantially flat as shown in FIGS. 2A and 2B said angle α is from about 35° to about 55°, more preferably from about 35° to about 50°. Preferably, a second fin (71) projects from said internal surface (101) of said plate (10) independently from said first fin (70), as shown in FIGS. 2A and 2B, and wherein said second fin (71) forms independently from said first fin (70) an angle β of from 15° to 75°. Said second fin (71) extends independently from said first fin (70) for an average second length (L2) of from about 2 mm to about 30 mm. Preferably, when said internal surface (101) of said plate (10) is substantially flat, said angle β is from about 35° to about 55°, more preferably from about 35° to about 50°. More preferably, said internal surface (101) of said plate (10) is substantially flat and said first (70) and second fin (71) form with said substantially flat internal surface (101) substantially identical angles α and β of from about 35° to about 55°. Preferably said first (70) fin and said second (71) fins project independently from said internal surface (101) of said plate (10) and extend with substantially identical average first and second lengths (L1; L2).

Each of said first (70) and second (71) fin has a distal edge (80; 81) and a proximal edge (90; 91). Said proximal edge (90; 91) are those attached to said internal surface (101) of said plate (10) as shown in FIG. 2A. Said proximal edges (90; 91) are each independently delimited by an average width (W1) for said first fin (70) and an average width (W2) for second fin (71) and each independently by average thickness (T1) for said first fin (70) and an average thickness (T2) for said second fin (71). Said average width (W1) and (W2) are preferably of from about 20 cm to about 0.5 cm, more preferably from about 15 cm to about 1.0 cm and even more preferably from about 10 cm to about 1.5 cm. Said average thicknesses (T1) and (T2) are preferably from about 5 mm to about 0.1 mm, more preferably from about 4 mm to about 0.5 mm, even more preferably from about 3 mm to about 0.5 mm. Preferably, said distal edges (80; 81) have also substantially identical average widths (W1) and (W2) and substantially identical average thicknesses (T1) and (T2) as those proximal edges (90; 91). Said first and second fins (70; 71) may have protrusions (75) as shown in FIG. 5P or may be embossed, especially to provide visual or tactile decoration.

Said first and second fin (70; 71) may project from said internal surface (101) of said plate (10) in any orientation one with respect to the other. In one embodiment said first and second fins (70; 71) and their proximal edges (90; 91) are curved as shown in FIG. 5N. Preferably, said first (70) and second (71) fin project from said internal surface (101) so that said proximal edge (90) of said first fin (70) is substantially parallel to said proximal edge (91) of said second fin (71) as shown in FIG. 2A. Preferably said proximal edge (90) of said first fin (71), more preferably said proximal edge (90) of said first fin (71) and said proximal edge (91) of said second fin (71) project from said internal surface (121) parallel to said axis Y of said plate (120) as shown in FIGS. 2A and 2B.

When said applicator (1) comprises a first and a second fin (70; 71) and irrespectively whether said proximal edges (90; 91) of said first and second fin (70; 71) project independently from said internal surface (101) of said plate (10) in a parallel fashion, said distal edge (80) of said first fin (70) and said distal edge (81) of said second fin (71) may verge one toward another as shown in FIG. 2B, may diverge toward opposite direction or they may point toward the same direction without verging.

In one embodiment of said applicator (1) according to the invention, said applicator (1) comprises a first fin (70) and a second fin (71), said first and second (70; 71) extend independently for substantially identical average lengths (L1) and (L2), said internal surface (101) of said plate (10) is substantially flat and said first fin (70) and said second fin (71) form with said substantially flat internal surface (101) substantially identical angles α and β of from about 35° to about 55°, said proximal edge (90) of said first fin (71) and said proximal edge (91) of said second fin (71) project from said substantially flat internal surface (101) parallel to said axis Y of said plate (10) and said distal edges (80; 81) verge one toward the other as shown in FIG. 2A. The selection of the orientation of the first and second fins to one another enables the applicator to be provided such that it can be used in multiple directions or in a single direction by the user.

Said hair orientation means may be provided in a variety of materials as previously described herein to manufacture the plate (10) or well (20) and be manufactured independently of said applicator (1). Preferably said hair orientation means is fluid impervious. More preferably said hair orientation means is selected from polyolefins, thermo plastic elastomers and mixtures thereof. In certain embodiments, both the said first (70) and second (71) fin and said plate (10) may be manufactured within the same injection or co-injection mould for example from thermo plastic elastomer.

### 3. Fluid Metering Means

The applicator (1) according to the present invention is characterized by a fluid metering means, comprising a first metering layer (50) and a second metering layer (60) as described herein. Said fluid metering means is selected from a group consisting of non-wovens, foams and combinations thereof. Said first metering layer (50) is positioned upon said rim (222) of said well (20). Said second metering layer (60) is positioned upon said internal surface (101), more preferably along said perimeter (103) of said plate (10).

Without wishing to be bound by theory, it is believed that by having said fluid metering means comprising a first metering layer (50) laid upon said rim (222) of said well (20) and a second metering layer (60) laid upon said internal surface (101) of said plate (10), preferably along said perimeter (103), whereby said first metering layer (50) and said second metering layer (60) are substantially juxtaposed when said applicator (1) is in a closed state, allows said applicator (1) not only to apply the hair treatment composition, but to even said application along the entire hair strand. Evenness is important in the application of a hair treatment composition, especially when said hair treatment composition is a highlighting composition or a dyeing composition. The permanent effect provided by those compositions is not immediately visible after the application and if the result is not appealing, it is not easily reversed. An applicator should hence ensure homogeneous application along the length, from root to tip, of said bundle of hair strands and likewise also on the front and rear surfaces and across the width of the bundle of hair strands. Therefore, it is not only the amount of hair treatment composition which is applied that is important but also the way it is applied. The applicator (1) according to the invention is not only designed to facilitate the application of a hair treatment composition to a hair strand, preferably to a bundle of hair strands. Said applicator (1) also avoids that said hair treatment composition is neither applied in excessive amount nor removed from said hair strand while the applicator (1) is used, so to obtain a very homogeneous and reproducible application.

The fluid metering means comprising a first (50) and second (60) metering layer are preferably independently selected from the group of non-wovens, foams or combinations thereof. More preferably the fluid metering means have a specific caliper and compressibility, as defined hereinafter.

The caliper measures the thickness of said fluid metering means and determines how the fluid metering means interacts with the hair strand. Each of said first (50) and second (60) metering layer has independently an average caliper, wherein the sum of said first (50) and second (60) average calipers provides the calculated thickness of said fluid metering means. Given that the average distance (D1) of the plate separation may vary between applicators of the current invention, and that the first (50) and second (60) metering layers need to be substantially juxtaposed when the applicator is in a closed state, the desired thickness of the fluid metering means depends on the average distance (D1) of the plate separation. Preferably the ratio of the thickness of the fluid metering means and the average distance is defined by the mathematical relationship:

4.375×D1≧thickness of fluid metering means≧0.792×D1

The capability of said hair strand to fit between said plate (10) and said well (20) is not only related to the thickness of the fluid metering means, but is also related to the ability of the first (50) and second (60) metering layers to compress under a specific force and for a defined time range.

Without wishing to be bound by theory, it is believed that a fluid metering means having a first (50) and second (60) fluid metering layers preferably having a specific compressibility facilitate the application of a hair treatment composition, with an applicator (1) according to the invention, to a hair strand in an even fashion avoiding scraping off or wiping away the just applied hair treatment composition.

Preferably, each of said first (50) and second (60) metering layer have a compressibility at about 0.5 k-Pa of from about 59% to about 93%. Said compressibility is preferably from about 60% to about 85%, more preferably from about 60% to about 77% as determined in the test methods hereinafter.

Said first metering layer (50) and said second metering layer (60) preferably have a substantially identical caliper and/or substantially identical compressibility as defined herein.

Said first metering layer (50) may be laid upon said rim (222) in a continuous or discontinuous manner. By discontinuous is meant that said metering layer may form loci or islets or may be interrupted. In one embodiment, said first metering layer (50) is laid upon the entire rim (222) of said wall (202) so that said rim (222) is not visible and entirely covered by said first metering layer (50). In another embodiment, said first metering layer (50) is laid upon only a portion of said rim (222), for example only a portion of said length of said rim (222) may comprise said first metering layer (50) as shown in FIG. 2A. Preferably said first metering layer and said second metering layer (60) are positioned adjacent to said connection (30). In each embodiment however the first metering layer (50) and the second metering layer (60) are positioned such that when said applicator is in a closed state said first metering layer (50) and said second metering layer (60) are substantially juxtaposed. The term juxtaposed as used herein includes embodiments wherein either of said first (50) or second metering layer (60) is larger than the other layer.

When said applicator (1) according to the invention comprises a member (40) as a hair orientation means said first metering layer (50) is laid upon said rim (222) and said second metering layer (60) is laid upon the internal surface (101) of said plate (10) to be in correspondence to said member (40) as explained herein below. When said applicator (1) comprises a member (40) for example as shown in FIG. 3, said member (40) extends along said internal surface (101) with a maximum length (L). The application of a hair treatment composition with an applicator (1) according to the invention is performed by locating said hair strand between said plate (10) and said well (20) and preferably said hair strand is located substantially transversal to said maximum length (L) of said member (40). To achieve an even application, said first metering layer (50) is preferably laid upon a portion of said rim (222) and said second metering layer (60) is laid upon a portion of the internal surface (101) which are substantially parallel to said maximum length (L) of said member (40) when said applicator is in a closed state, and whereby said first and second metering layers (50;60) are substantially juxtaposed.

In another embodiment of the present invention as shown in FIG. 2A, said hair orientation member comprises a first and a second fin (70; 71) as said hair orientation member (2). Said proximal edges (90; 91) of said first and second fins (70; 71) are substantially parallel to said axis Y of said plate (10). In this embodiment said first metering layer (50) is preferably laid upon a portion of said rim (222) and similarly said second metering layer (60) which are substantially parallel to said proximal edges (90; 91) of said fins (70; 71) when said applicator (1) is in a closed state.

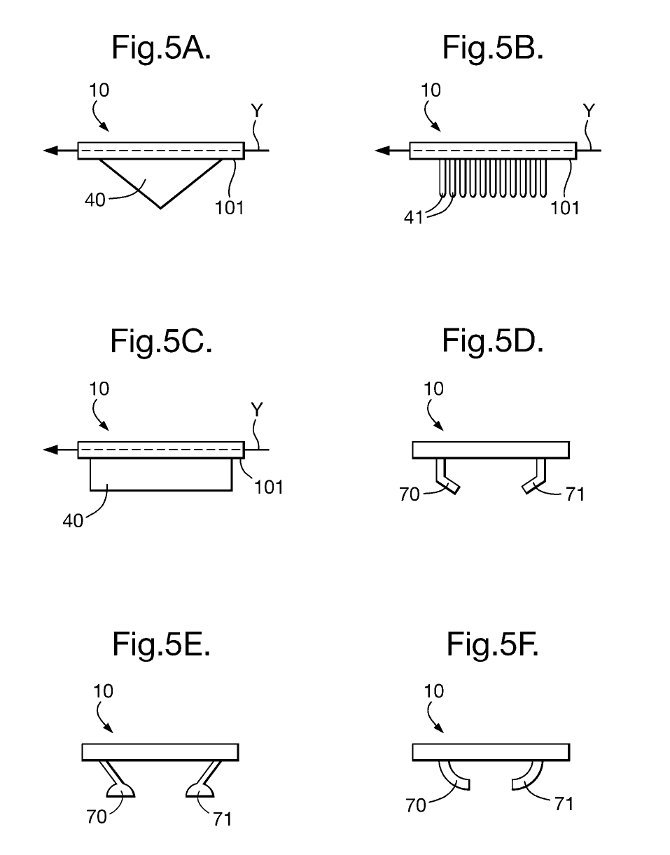

Clearly, for both the embodiments discussed above, said first (50) and second (60) metering layers may be discontinuous (50; 50′), (60;60′) and laid upon said rim (222) of said wall (222) and said internal surface (101) of said plate (10) respectively on both sides parallel to said maximum length (L) of said member (40) or of said distal edges (90; 91) of said first and second fins (70; 71), as shown in FIG. 4.

Preferably, when an applicator (1) according to the invention comprises a sealing means (401) as described below, on said rim (222) of said well (202), said first metering layer is laid upon said rim (222) adjacently to said sealing means (401) either touching said sealing means (401) or not.

In one embodiment said second metering layer (60) may be laid upon said entire internal surface (101) of said plate (10). Said second metering layer (60) may be laid upon said hair orientation means such as a member or fins preferably, said second metering layer (60) is not laid upon said hair orientation means.

In another embodiment said second metering layer (60) is laid upon only a portion of said internal surface (101), preferably along said perimeter (103) of said plate (10).

When said applicator (1) comprises a first metering layer (50) and said second metering layer (60) is laid upon said internal surface (101) of said plate (10) so that when said internal surface (101) said plate (10) is brought into a juxtaposed relationship to said opening (203) of said well (20), said second metering layer (60) is juxtaposed to said first metering layer (50).

Said first and second metering layers (50; 60) may independently have a length of from about 3 mm to about 40 cm, preferably from about 5 mm to about 10 cm, more preferably from about 8 mm to about 5 cm.

Said first and second metering layers (50; 60) may independently have a constant or variable width along said lengths. Said first and second (50; 60) metering layers may independently have a width of from about 1 mm to about 20 mm, preferably from about 2 mm to about 15 mm, more preferably from about 3 mm to about 8 mm. In addition, said first (50) and second (60) metering layers may have constant or variable calipers along their lengths.

Preferably, said first and second metering layers (50; 60) have substantially identical widths and substantially identical lengths and they are laid upon said rim (222) and said internal surface (101) along said perimeter (103), respectively, so that when said plate (10) is brought into a juxtaposed relationship to said opening (203) of said well (20), said second metering layer (60) is substantially a mirror image of said first metering layer (50). Even more preferably said first and second metering layer (50; 60) have substantially identical and constant widths, lengths and calipers.

Said first (50) and second (60) metering layers are selected from the group consisting of non-wovens, foams and combinations thereof.

Suitable non-wovens may be comprised of natural or synthetic fibers selected from acetate fibers; acrylic fibers; cellulose ester fibers; modacrylic fibers; polyamide fibers; polyester fibers; polyolefin fibers; polyvinyl alcohol fibers; rayon fibers; polyethylene foam; keratin fibers; cellulose fibers; silk fibers and combinations thereof. The non-wovens may be comprised of mono-component fibers, such as a polyolefin or polyester, or bi-component fibers, such as a sheath/core fiber or side by side fiber of polyethylene/polypropylene or polyethylene/polyester, or bi-constituent fibers comprised by a blend of two or more thermoplastic polymers.

Examples of suitable Carded non-wovens include; PGI 214 and Libeltex 01-766 DI-4. Further examples of suitable non-wovens include USFELT F-50 and Ahlstrom 18008.

Foam materials are made from low density elastomers, plastics, and other materials with various porosities and may be selected from open cellular foams; flexible foams and reticular foams and syntactic foams which can be fabricated into finished shapes using molding, casting, extrusion, pultrusion, machining, thermal forming, plastic welding, blow molding, rapid prototyping techniques, grinding and/or other specialized processes. The foam materials may be composed of a variety of chemical systems including Acrylonitrile-Butadiene-Styrene; Acrylics; Epoxy resins; Fluoropolymers; isoprene-styrene and Styrene-Butadiene-Styrene; Synthetic rubbers or elastomers based on a variety of systems such as silicone, polyurethane, polyolefin and neoprene; Nitrile rubbers; plastics or elastomers formed from natural or plant-based raw materials such as natural rubber (polyisoprene) or vulcanized fibre; water-based and water-borne resins and latex materials. Chemical systems for foams may include ethylene copolymer, expanded polyethylene, polycarbonate, polyester, polyether, polyetherimide, polyimide, polyolefin, polypropylene, phenolic, polyurea, and vinyl. Examples of suitable foams include; Recticel Bulpren D32133; Recticel D27150 B and Recticel Bulpren S31048

The fluid metering means of the present invention may also include composite materials having one or more plies of the same or different materials superimposed physically, joined together continuously (laminated), in a discontinuous pattern, or by bonding the external edges at discrete loci.

Said first and second metering layer (50; 60) may be attached by any suitable method to said rim (222) and to said internal surface (101) of said plate (10), respectively, providing that said method does not destroy or alter the performance of said metering layers (50; 60).

The present inventors have surprisingly found that to satisfactorily apply a hair treatment composition to a hair strand, said hair strand needs not only to come into contact with said hair treatment composition which has been loaded into said applicator (1), but also said hair treatment composition should be evenly applied onto said hair strand without being removed while the application occurs.

To apply a hair treatment composition with an applicator (1) to a hair strand, preferably to a bundle of hair strands, said hair strand is located substantially straight between said plate (10) and said well (20), where a hair treatment composition has been previously loaded. Said plate (10) comprises a hair orientation means to bend said hair strand into said well (20).

Without wishing to be bound by theory, it is believed that when said applicator (1) comprises a fluid metering means as described herein, a hair treatment composition is more evenly distributed from root to tip along a bundle of hair strands, and likewise also on the front and rear surfaces and across the width of the bundle of hair strands. Thus, if an excessive amount of said hair treatment composition is applied, said metering means may distribute it evenly and homogeneously along the length of said hair strand but without removing it.

### 4. Additional Features

The applicator (1) may further comprise one or more sealing means, preferably one sealing means (401) is present within the hair treatment applicator (1). Said sealing means (401) may be located within said well (20) on said bottom (201) at the wall (202) adjacent to said connection (30) as shown in FIG. 3. Said sealing means (401) may be located on the bottom (201) of said well (20) adjacent to said wall (202) at the side of the connection (30) or on said internal surface (101) adjacent to said connection (30). Preferably said sealing means (401) is part of said connection (30).

The sealing means (401) is provided to avoid displacement of hair treatment composition towards the connection (30) and hair from being trapped within said connection (30) when said internal surface (101) of said plate (10) is brought into a juxtaposed relationship to said opening (203) of said well (20). Useful materials to manufacture a sealing means (401) include those described herein for said first fin (71) and foams.

One or more stop mechanisms may be incorporated onto said applicator (1). The stop mechanism collaborates with said connection (30) to ensure that when said internal surface (101) of said plate (10) is brought into a juxtaposed relationship to said opening (203) of said well (20), the average distance (D1) between said perimeter (103) and said rim (222) is controlled. In one embodiment as shown in FIG. 1A, two stop mechanisms are comprised on said rim (222) of said wall (201) of said well (20), preferably said two stop mechanisms are two substantially identical hemispheres (402; 403). Useful materials to manufacture a stop mechanism (402) are previously described herein to manufacture the plate (10) or well (20) described herein.

The applicator (1) disclosed herein may further comprise gripping areas on the external surfaces (102) of said plate (10) and/or on said bottom (201) of said well (20). Said gripping areas are designed to provide grip. Gripping means may be provided as fastening means to accommodate the user's fingers.

Fingers may be used to select the hair strands on which the hair treatment composition should be applied. The applicator (1) of the present invention may however be further provided with hair strand selection means. Examples of hair strand selection means are, but not limited to, spikes, hooks, crochets, clips or beads. The hair strand selection means may be incorporated onto said plate (10) and/or said well (20). Said means may also be attached through a snap mechanism to said plate (10) and/or said well (20) such that the hair strand selection means may swing from a position proximal to said plate (10) and/or said well (20) to a far one, such as happens with the blades of a penknife. The hair strand selection means may also be separately provided to the applicator (1) of the present invention as a component of a kit as described herein below.

### 5. Method of Use

The present invention also relates to a method to apply a hair treatment composition with said applicator (1) according to the invention to a hair strand, preferably a bundle of hair strands, wherein said applicator (1) comprises said hair treatment composition and whereby said hair strand is contacted with said applicator (1). Said applicator (1) may be pre-loaded with one or more hair treatment compositions, but preferably one or more hair treatment compositions are loaded into said applicator (1) before the contact of said hair strand, preferably said bundle of hair strands, with said applicator (1).

The hair treatment compositions may be a single hair treatment composition or may be formed by a first hair treatment composition which requires mixing with a second hair treatment composition before application to the hair. Preferably, said first and second hair treatment compositions are mixed to form a third hair treatment composition. Said third hair treatment composition is loaded in said hair treatment applicator (1) before contacting the hair strand, preferably a bundle of hair strands, with said hair treatment applicator (1).

Once the hair treatment applicator (1) is loaded with one or more hair treatment compositions, the user holds through the external surfaces (102) of said plate (10) and bottom (201) of said well (20) of said applicator (1) in one hand, preferably between the thumb and the index finger. Once the user has selected the hair strands to be treated, said hair strand, preferably said bundle of hair strands, is located between said plate (10) and said containment portion (20) while the applicator (1) is in an open state. Subsequently said internal surface (101) of said plate (10) is brought into a juxtaposed relationship to said opening (203) of said well (20). Said applicator (1) is swiped along the length of said hair strand, preferably on said bundle of hair strands, and one or more hair treatment compositions are applied. More preferably, said hair treatment applicator (1) is located at the root-line of said hair strand, preferably at the root-line of said bundle of hair strands. The hair treatment composition may also be applied only to limited areas of the hair, i.e. the user can coat only the root-line with the hair treatment composition. The swiping may be repeated more than once, preferably twice.

Finally, the application of the hair treatment composition may occur on wet or dry hair and optionally, a rinsing or a shampooing step can be included between application of the first and second compositions to the hair.

### 6. Hair Treatment Compositions, Use Thereof and Kit

The present invention further comprises a kit. Said kit comprises an applicator (1) according to the invention and one or more individually packaged hair treatment compositions. Preferably, these compositions are selected from the group consisting of styling compositions, dyeing compositions, highlighting compositions or combination thereof. Each of these hair treatment compositions or combinations thereof may be used to provide a hair strand effect with said applicator (1) described above. Preferably said one or more hair treatment compositions have a rheology of from about 10 Pa to about 160 Pa, more preferably of from about 12 Pa to about 120 Pa, most preferably from 15 Pa to 80 Pa at 1 s⁻¹. More preferably, said one or more hair treatment composition is a highlighting composition.

The rheology of the hair treatment composition is measured using a TA Instruments Advanced Rheometer (AR) 2000. The instrument is provided with a concentric cylinder base with an internal radius of 15.00 mm and standard size vane geometry with a radius of 14.00 mm and a height of 42.00 mm. The geometry gap is set at 4000 microns. Hair treatment compositions which are made up of more than one formulation are prepared by mixing those various formulations thoroughly by hand shaking in a sample pot for 30 seconds. The mixed hair treatment composition is then placed immediately into the concentric cylinder base, and the standard vane geometry is lowered to the geometry gap such that the top of the vanes are covered by the hair treatment compositions. The temperature is equilibrated to 25° C., and then hair treatment composition is left for an additional 30 seconds before the shear rate increases logarithmically from about 0.05 to about 200 s⁻¹, recording seven points per decade. At all stages the temperature is maintained at 25° C. The shear stress is recorded at 1.0 s⁻¹ and reported in Pa.

Examples of hair treatment compositions which can be used with the hair treatment applicator (1) according to the invention are indicated below in table 1.

The hair treatment compositions may comprise components known, conventionally used, or otherwise effective for use in hair treatment compositions particularly oxidative bleaching and dye compositions which include but are not limited to: developer dye compounds; coupler dye compounds; direct dyes; oxidizing agents; reducing agents; thickeners; chelants; pH modifiers and buffering agents; alkalising agents, carbonate ion sources and radical scavenger systems; glycine; amodimethicone, ethylenediamine disuccinic acid; anionic, cationic, non-ionic, amphoteric or zwitterionic surfactants, or mixtures thereof, anionic, cationic, non-ionic, amphoteric or zwitterionic polymers, hydrophobically modified polymers or mixtures thereof, fragrances; dispersing agents; solvents, peroxide stabilizing agents; chelants, humectants, proteins and derivatives thereof, plant materials (e.g. aloe, chamomile and henna extracts); silicones (volatile or non-volatile, modified or non-modified), film-forming agents, cellulose polymers and their derivatives, ceramides, preserving agents, gel networks, colour indicators and opacifiers. Some adjuvants which are suitable are listed in the International Cosmetics Ingredient Dictionary and Handbook, (8th ed.; The Cosmetics, Toiletry, and Fragrance Association). Particularly, vol. 2, sections 3 (Chemical Classes) and 4 (Functions) are useful in identifying specific adjuvants to achieve a particular purpose or multipurpose. A representative but not exhaustive list of polymers and thickening agents can be found in “The Encyclopaedia of Polymers and Thickeners for Cosmetics” compiled and edited by Robert Y. Lochhead, PhD and William R. Fron, Department of Polymer Science, University of Southern Mississippi.

The present invention further comprises a kit. Said kit comprises an applicator (1) as described above and one or more individually packaged hair treatment compositions. More than one applicator (1) may be comprised in said kit.

In one embodiment of the present invention, said one or more individually packaged hair treatment compositions comprise a first individually packaged hair treatment composition and a second individually packaged hair treatment composition. When mixed said first and second individually packaged hair treatment compositions form a third hair treatment composition. Examples of such compositions include so called semi-permanent and permanent colorants which typically contain oxidative dyes and an oxidant, and highlighting compositions containing an oxidant and an alkalising agent, optionally with a persulfate salt. Preferably, said first individually packaged composition comprises an oxidizing agent and said second individually packaged composition comprises an alkalizing agent. Preferably, said oxidizing agent is hydrogen peroxide. More preferably, at least one of said first and/or second individually packaged hair treatment composition comprises a persulfate salt.

In one embodiment of the kit according to the present invention said first individually packaged hair treatment composition comprises from 3% to 12% of hydrogen peroxide by weight of said first individually packaged hair treatment composition and said second individually packaged hair treatment composition is in the form of a powder or paste activator and said second individually packaged hair treatment composition comprises from 10% to 60% of persulfate salt selected from sodium persulfate, potassium persulfate, ammonium persulfate or mixtures thereof, by weight of said second individually packaged hair treatment composition. Said kit optionally comprises a third individually packaged hair treatment composition comprising from 3% to 25% of an alkalizing agent in an aqueous vehicle, by weight of said third individually packaged hair treatment composition.

In another embodiment of the present invention said first individually packaged hair treatment composition comprises from 1.5% to 12% of hydrogen peroxide by weight of said first individually packaged hair treatment composition and said second individually packaged hair treatment composition comprises from 0.01% to 6% of a dye selected from direct dyes, oxidative dye precursors, oxidative dye couplers or mixtures thereof, by weight of said second individually packaged hair treatment composition.

Additional individually packaged hair treatment compositions may be present in the kit and may comprise shampoos, conditioner or styling products.

Herein below in are given some examples of hair treatment compositions which may be loaded into the hair treatment applicator (1) according to the invention.

A hair bleaching composition was prepared by mixing about 45 g of any of the formulations of Phase 1 (1.1, 2.1, 3.1, 4.1, table 1), which were in a liquid form with about 15 g of any of the formulations of Phase 2 (1.2, 2.2, 3.2, 4.2, in table 1), which were in a powder form. Mixing was achieved as follows: the powder formulation of Phase 2 was placed into a mixing tray and the liquid formulation of Phase 1 was poured on top of the powder. The two formulations were then mixed together using a spatula to form a bleaching composition. Mixing was completed when the bleaching composition looked visually homogeneous.

TABLE 1
Formulations of Phase 1 and 2 which can be mixed to form a highlighting composition.
All ingredients are in percentage by weight of the formulation phase.

| | Phase 1 | | | |
|---|---|---|---|---|
| | 1.1 | 2.1 | 3.1 | 4.1 |
| De-ionized Water | q.s. to 100% | q.s. to 100% | q.s. to 100% | q.s. to 100% |
| Glycerine | | | | |
| Hydrogen Peroxide | 17.2 | 17.2 | 17.2 | 17.2 |
| (35% Active) | | | | |
| Disodium EDTA | 0.04 | 0.04 | 0.04 | 0.04 |
| Sodium Hydroxide | q.s. to pH 3.5 | q.s. to pH 3.5 | q.s. to pH 3.5 | q.s. to pH 3.5 |
| (50% aq. Solution) | | | | |
| Stearyl Alcohol ¹ | 2 | 1 | 2 | 2 |
| Cetyl Alcohol ² | 3 | 1.5 | 3 | 3 |
| Ceteareth 25 ³ | 1.5 | 0.75 | 1.5 | 1.5 |
| Aculyn ™ 33 ⁴ | | 2.4 | | |
| Salcare ™ SC 90 ⁵ | | | 1 | |

Phase 2

| Persulfate Powders | 1.2 | 2.2 | 3.2 | 4.2 |
|---|---|---|---|---|
| Ammonium Persulfate | 28.6 | 28.6 | 28.6 | 28.6 |
| Potassium Persulfate | 50 | 50 | 50 | 47 |
| Sodium Persulfate | 7.14 | 7.14 | 7.14 | 7.14 |
| Sodium Metasilicate | 14.26 | 14.26 | 14.26 | 14.26 |
| Carbopol ™Ultrez 10 ⁶ | | | | 3 |

¹ Stearyl Alcohol Crodacol S-95, Croda, Inc.
² Cetyl Alcohol, Crodacol C-70, Croda, Inc.
³ Cetearth 25, Cremophor A 25, BASF Corporation
⁴ Aculyn ™ 33, Rohm and Hass Company Inc.
⁵ Salcare ™ SC 90 Ciba Specialty Chemicals Corporation
⁶ Carbopol ™Ultrez 10

### 6. Test Methods

Caliper

The caliper of a metering layer was determined using the general procedure described in “ASTM D 5736-95 Standard Test Method for Thickness of Highloft Non-Woven Fabrics”. A die cutter was used to prepare circular samples of metering layer of about 35.7 mm in diameter. Care was taken to avoid compression and/or disturbance of the metering layer during handling. Any metering layer with defects, such as folds, wrinkles, non-uniformity, creases or cut marks etc, were rejected from testing. The caliper was measured on a TA Instruments Ltd DMA 2980 with two parallel circular plates of 40 mm in diameter (compressive plates—anvil and presser foot) setup and calibrated in the compressive plates mode according to the manufacturer's guidelines. The opposing flat surfaces of the two parallel plates were brought in contact from their resting position and the dimensional change was manually zeroed. The plates were restored to their resting position and the metering layer to be tested was centrally positioned on the surface of the lower plate avoiding any compression and/or disturbance to the substrate during handling. The preload force was set to zero Newton. A 0.02 kPa pressure was applied to the sample in 5 seconds and the pressure held constant for an additional 10 seconds. The caliper was recorded at a time between 9 to 10 seconds after the 0.02 kPa had been reached. The measurement was repeated three times for each metering layer on a new sample. The average caliper at 0.02 kPa was defined as being the average caliper and was recorded to the nearest 0.001 mm. The same method was applied to measure the caliper of metering layer non-wovens, foams and combinations thereof. Any equipment capable of measuring the caliper of the metering layer according to the procedure described herein above can be used.

Compressibility

The compressibility of a metering layer was determined as the percentage compressibility according to the following equation (I)

$$\% Compressibility = 100 \times \frac{Calliper at 0.5 kPa}{Calliper at 0.02 kPa} \qquad ( I )$$

The caliper of the metering layer was first determined at a pressure of 0.02 kPa as described above on a TA Instruments Ltd DMA 2980. The caliper was recorded at a time between 9 to 10 seconds after the 0.02 kPa had been reached. A pressure of 0.5 kPa was then applied to the same sample in 5 seconds and the pressure held constant for an additional 10 seconds. After the pressure was held constant the caliper at 0.5 kPa was recorded at a time between 9 to 10 seconds. The measurement was repeated three times for each metering layer on a new sample. The average caliper at 0.5 kPa was recorded to the nearest 0.001 mm and the percentage compressibility was calculated according to the equation (I) above. The same method was applied to measure the caliper of metering layers selected from non-wovens, foams or combinations thereof. A pressure of 0.5 kPa was chosen to measure the caliper of the metering layers to determine their compressibility. This represents a meaningful pressure that consumers may apply to the applicator (1) according to the invention. In addition a pressure of 0.5 kPa allows the compressibility of different metering layers to be measured from one another. Any equipment capable of measuring the caliper of the metering layer according to the procedure described herein above can be used.

Average Distance (D1)

The average distance (D1) between said perimeter (103) of said plate (10) and said rim (222) of said well (20) was measured when said applicator is in a closed state and said internal surface (101) of said plate is in a juxtaposed relationship to said opening of said well (20). Mitutoyo Digimatic calipers were positioned at said perimeter (103) and the distance from said perimeter (103) to said rim (222) was measured. This measurement was repeated for another ten positions around the perimeter (103) and the rim (222). Said ten positions were taken as each equally spaced along the length of said rim (222) of said well (20). The ten measurements were averaged to provide the average distance (D1). If any stop mechanism is located at the perimeter (103) of said plate (10) it is not comprised within the measurement as not been part of said perimeter (103).

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm”.
1 Applicator
10 Plate
11 strip
12 strip
20 Well
30 Connection
32 connection female part
34 connection female part
38 connection pin
40 Member
41 plurality of tines
49 1^{st} slot
50 First metering layer
50′ First metering layer (second part)
59 2^{nd} slot
60 Second metering layer
60′ Second metering layer (second part)
70 First fin
71 Second fin
75 fin protrusions
80 distal edges
81 distal edges
90 proximal edges
91 proximal edges
101 Internal Surface
102 External Surface
103 Perimeter
201 well bottom
202 Wall
203 Opening
204 internal volume
222 Rim
401 sealing means
402 stop mechanism
403 stop mechanism
α first fin angle
β second fin angle
L1 first fin length
L2 second fin length
W1 first fin width
W2 second fin width
T1 first fin thickness
T2 second fin thickness
D1 Plate separation
L member length
H member height
W member width

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm”.

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments or the present invention have been illustrated and described, it would be obvious to those skilled in the art that various other changes and modifications can be made without departing from the spirit and scope of the invention. It is therefore intended to cover in the appended claims all such changes and modifications that are within the scope of this invention

What is claimed is:

1. An applicator for applying a hair treatment composition to the hair, wherein said applicator comprises
a plate and a well, wherein said plate and well are movably joined by a connection so that the applicator may alternate between a closed and an open state, and
wherein said plate has an external surface and an internal surface, and said internal surface comprises at least one hair orientation means which extends beyond said internal surface towards said well, and
wherein said well comprises a rim and
wherein said applicator further comprises a fluid metering means, wherein said fluid metering means comprises a first metering layer positioned on said rim of said well, and a second fluid metering layer positioned on said internal surface of said plate,
such that when said applicator is in said closed state said first metering layer and said second metering layer are substantially juxtaposed to provide said fluid metering means.

2. The applicator according to claim 1, wherein said hair orientation means is fluid impervious.

3. The applicator according to claim 1, wherein said hair orientation means comprises a first fin and a second fin.

4. The applicator according to claim 1, wherein said hair orientation means is selected from polyolefins, thermo plastic elastomers and mixtures thereof.

5. The applicator according to claim 1, wherein said first metering layer is substantially a mirror image of said second fluid metering layer.

6. The applicator according to claim 1, wherein said fluid metering means is selected from nonwovens, foams and combinations thereof.

7. The applicator according to claim 1, wherein said first metering layer and said second metering layer are positioned adjacent said connection.

8. The applicator according to claim 1, wherein said first metering layer and said second metering layer are independently discontinuous.

9. A method to apply a hair treatment composition with said applicator according to claim 1, to a hair strand, wherein said method comprises applying a hair treatment composition to said applicator in said open state, selecting a bundle of hair strands, placing said hair strands in said applicator and bringing said applicator into said closed position and then swiping said along the length of said hair strand.

10. A kit-of parts comprising an applicator according to claim 1; and one or more individually packaged hair treatment compositions, and instructions for use.

11. Use of one or more hair treatment compositions or combinations thereof with an applicator according to claim 1 to provide a hair strand effects.
